(19) United States
(12) Patent Application Publication
(10) Pub. No.: US 2014/0155937 A1
(43) Pub. Date: Jun. 5, 2014

(54) BRIDGE BUTTON FOR LIGAMENT RECONSTRUCTION

(71) Applicant: Padmakar Shinde, Nashik (IN)

(72) Inventor: Padmakar Shinde, Nashik (IN)

(21) Appl. No.: 13/979,051

(22) PCT Filed: Jan. 11, 2011
(86) PCT No.: PCT/IN12/00030

§ 371 Date: Nov. 26, 2013

(51) Int. Cl. A61B 17/04

(52) U.S. Cl. CPC A61B 17/0401
USPC 606/232

(57) ABSTRACT

A bridge button formed by an elongate body of pre-defined dimensions such that said button being adapted to sit across a cavity in a bone, said button comprising a span member, located substantially at the centre of said elongate body, adapted to define a bridge portion, said span member being defined by lateral edges of sutures placed across adjacent inner circumferences of a pair of pre-determined holes used for passing suture loop, along a longitudinal axis of said bridge button, for holding a graft.

### FIELD OF THE INVENTION

This invention relates to the field of biomedical engineering.

Particularly, this invention relates to the field of biomedical engineering related to ligament reconstruction.

Still particularly, this invention relates to mechanical fixtures for ligament reconstruction.

More particularly, this invention relates to a multipurpose bridge button used for cortical blowouts in for ligament reconstruction as well as for a novel Combihole technique for Double bundle ACL reconstruction.

### BACKGROUND OF THE INVENTION

Knee, in humans, support the entire body weight. It is hence susceptible to injury, apart from wear and tear. The knee is the largest joint in the human body. The knee joint joins the thigh with the leg and consists of two articulations: one between the femur and tibia, and one between the femur and patella. It provides flexion and extension movement apart from slight medial and lateral rotation.

The components of the knee include ligaments; which offer stability by limiting movements. Cruciate ligaments are ligaments which cross each other like the letter ‘X’. Although, they allow a large range of motion, they stabilize the knee. The cruciate ligaments of the knee are the anterior cruciate ligament (ACL) and the posterior cruciate ligament (PCL). The ACL is lateral and the PCL is medial.

The ACL originates from deep within the notch of the distal femur. Its proximal fibers fan out along the medial wall of the lateral femoral condyle. There are two bundles of the ACL—the anteromedial and the posterolateral, named according to where the bundles insert into the tibial plateau. The ACL attaches in front of the intercondyloid eminence of the tibia, being blended with the anterior horn of the lateral meniscus. These attachments allow it to resist anterior translation of the tibia, in relation to the femur.

Anterior cruciate ligament injury is the most common knee ligament injury, especially in athletes. Lateral rotational movements in sports like these are what cause the ACL to strain or tear. Anterior cruciate ligament (ACL) injury or Posterior Cruciate ligament (PCL) is normally treated by reconstruction which nowadays is done or assisted by arthroscopy. The ACL is the most commonly injured ligament of the knee and can be damaged in sports injuries or accidental injuries.

The ACL injury is followed by instability and repeated episodes of giving way which can damage the menisci and result in osteoarthritis or degeneration of the knee if left untreated.

The results of repair have been consistently unsuccessful; hence the ligament is replaced by various autologous grafts like the patellar tendon, hamstrings—Semitendinosus and/or the Gracilis, central third quadriceps or allografts. Recently, the hamstrings are becoming increasingly popular as their harvest does not follow morbidity.

The ACL has two distinct anatomic and functional bundles namely the Anteromedial (AM) bundle and the Posterolateral bundle (PL) named on the basis of their location on the tibia. The AM is the primary restraint against anterior translation of the tibia in flexion and the PL bundle is the primary restraint in extension. The two bundles cross each other in flexion with the AM bundle being posterior to the AL bundle in flexion and moving superior to the PL bundle in extension. In addition the ACL also provides rotational stability.

For reconstruction, two techniques are in use; namely:

(1) Single bundle ACL Reconstruction: where the native ACL is replaced by a quadrupled hamstring semitendinosus graft or doubled Semitendinosus-Gracilis graft which is attached to the femur and tibia through single tunnels made in the anatomic centre of the native ACL. This is, by far, the most commonly performed surgery and is simpler to perform than the double bundle technique.

(2) Double bundle ACL Reconstruction: since the native ACL has two different bundles namely the AM and the PL bundles which are taut in different flexion angles a single bundle does not restore the original anatomy of the ACL as well as a double bundle ACL reconstruction.

In this technique, two separate tunnels are drilled into the femur and the tibia in their anatomic centres and two separate grafts are used to recreate the two bundles; so also the two grafts are fixed separately with separate implants in the femur and tibia.

There are reports of residual instability and pivot shift following single bundle reconstruction with early degeneration of the joint. This can be prevented by double bundle reconstruction presumably though no long term studies or evidence is yet available.

In addition to this, the size of the native ACL varies considerably and the size of the graft may not match it in single bundle reconstruction if the native ACL has a very large footprint.

Various implants are in vogue for fixing the soft tissue graft at the femur but can be broadly divided into (1) Suspensory cortical fixation outside the tunnel e.g. the endobutton or the Transfix

(2) Aperture fixation e.g. with interference screw.

It is well settled that the suspensory fixation provides a very strong and secure femoral fixation.

On the tibial side the graft can be secured with interference screw or tied over a suture disc or over a suture post with screw and washer.

The reconstruction requires technically demanding steps like femoral and tibial drilling and in case of Endobutton CL fixation a stepped socket needs to be drilled in the femur without which fixation with endobutton is impossible. Problems like posterior tunnel wall blowout are common and can only be avoided with technical detail.

The PCL is an intracapsular ligament along with the anterior cruciate ligament (ACL) because it lies deep within the knee joint. They are both isolated from the fluid-filled synovial cavity, with the synovial membrane wrapped around them. The PCL gets its name by attaching to the posterior portion of the tibia.

The function of the PCL is to prevent the femur from sliding off the anterior edge of the tibia and to prevent the tibia from displacing posterior to the femur. Common causes of PCL injuries are direct blows to the flexed knee, such as the knee hitting the dashboard in a car accident or falling hard on the knee, both instances displacing the tibia posterior to the femur.

A torn anterior cruciate ligament cannot be “repaired”, and must instead be reconstructed with a tissue graft replacement.

For reconstruction, a hole is drilled through the femur and tibia. The graft forming the ligament is guided through the drill hole and attached in place on the external walls of the bones, typically by endobutton on the femur and sutures tied over a post on the tibia (suspensory fixation). Alternatively the grafts may be secured inside the tunnels at the apertures with bioabsorbable screws or metallic screws (aperture fixation) in order to complete the process of attachment.

Advances in arthroscopy have led to the design and availability of buttons which hold the graft and sit across the drilled hole in the form of an anchor. In the current form of surgery, a hole of a defined diameter is drilled through the medial side of the femur in a transverse direction. After reaching the midpoint of the femur, a narrower tunnel is drilled to complete the hole through to the lateral side of the bone. A button sits as an anchor on this lateral side, atop the cavity defined by the hole.

However, it has been observed that the anchor buttons available in the market work on the pre-condition that the hole is accurately drilled in accordance with specified parameters of dimensions.

Depending upon the numerous kinds of cases and bone structure and size, it becomes difficult for the surgeon to drill actual textbook holes, in spite of precision equipment. It has been observed that ‘blowouts’ may occur, rendering hole diameters larger that the length of the anchor buttons available to the surgeon.

Smith and Nephew® has provided an Endobutton® regular without suture loops and Endobutton CL® with prefabricated knotless polyester loop of sizes from 10 mm to 60 mm which is an anchor button which caters to holes up to 10 mm (4.5 mm) in size. In order to counteract the blowout condition, Smith and Nephew® developed an Xtendobutton® which is a larger anchor button which works in conjunction with the Endobutton®; and can cater to blowouts from 6 to 10 mm.

The Xtendobutton® displays potential for side to side movement resulting in potentially inadequate cover and hence insecure fixation in blowout holes: particularly the 10 mm hole.

There is a need for a reliable button (or anchorage device) which provides unconditional stability.

In cases of double tunnel reconstruction, the Double Bundle PCL Guides give versatility in creating appropriate socket placement using anatomical constants or directly visualizing the intended socket diameter with the guides. Two holes side by side form the double tunnel to receive the double bundle.

The grafts are passed through respective tunnels for securing.

### OBJECTS OF THE INVENTION

An object of the invention is to provide a reliable anchorage device for holding on to a graft.

Another object of the invention is to provide a reliable and stable anchorage device for holding on to a graft in the event of a blowout of a tunnel in the bone.

Still another object is to provide a simplified technique and implants for DBACLR

### SUMMARY OF THE INVENTION

For the purposes of this invention, a ‘button’ relates to an anchorage device adapted to provide anchor support to a ligament graft. Typically, the button sits across the outer surface of the cavity of a hole through which the graft is passed.

According to this invention, there is provided a button formed by an elongate body of pre-defined dimensions. The button body has pre-defined dimensions so that the button is adapted to sit across a cavity in a bone.

According to this invention, there is provided a bridge button formed by an elongate body of pre-defined dimensions such that said button being adapted to sit across a cavity in a bone, said button comprising a span member, located substantially at the centre of said elongate body, adapted to define a bridge portion, said span member being defined by lateral edges of sutures placed across adjacent inner circumferences of a pair of pre-determined holes used for passing suture loop, along a longitudinal axis of said bridge button, for holding a graft.

According to this invention, there is also provided a bridge button formed by an elongate body of pre-defined dimensions such that said button being adapted to sit across a cavity in a bone, said button comprising a span member, located substantially at the centre of said elongate body, adapted to define a bridge portion, said span member comprising an under-collar which is a protrusion of pre-defined height and pre-defined length in accordance with the bridge principle, said protrusion being located substantially at the centre of the anchorage device or button, said under-collar comprising lateral walls which are orthogonal to the operative bottom surface of said button.

According to this invention, there is further provided a bridge button formed by an elongate body of pre-defined dimensions such that said button being adapted to sit across a cavity in a bone, said button comprising a span member, located substantially at the centre of said elongate body, adapted to define a bridge portion, said span member comprising an under-collar which is a protrusion of pre-defined height and pre-defined length in accordance with the bridge principle, said protrusion being located substantially at the centre of the anchorage device or button, said under-collar comprising lateral walls which are orthogonal to the operative bottom surface of said button, said button still further comprising a recessed operative top surface, said recess being co-axial to any external button housing a graft.

Typically, at least a hole is provided on the protrusion of said button.

Typically, at least a hole is provided on the shoulders of said button.

Typically, said span member is a solid member, located substantially at the centre of said elongate body of said button, adapted to define a bridge portion.

Typically, said span member is defined in accordance with the cavity it is adapted to sit across.

Typically, said span member includes a plurality of holes for various purposes apart other than passing sutures for holding the graft.

Typically, said span member includes a plurality of holes at pre-determined locations.

Typically, said bridge button comprises a plurality of holes at pre-determined locations, beyond said bridge, adapted to allow passing of sutures for holding said graft, sutures for handling said button, sutures for handling an insert and the like purposes such that said holes may be selected in various combinations.

Typically, said bridge button comprises a first laterally located hole at a first longitudinal edge of the button for passing sutures which act as the pulling suture, said pulling suture aiding in pulling said button along with graft through a formed tunnel.

Typically, said bridge button comprises a second laterally located hole at a second longitudinal edge (longitudinal diametrically opposite to said first longitudinal edge) of the button used for passing sutures which act as the flipping suture, said flipping suture being engaged, by the surgeon, once the button has been pulled out of the tunnel on the lateral side of the bone, aiding in flipping (configuring) button from its operative vertical position (for pulling purposes) to its operative horizontal position wherein it can rest upon the cavity, transversely.

Typically, said button comprises holes placed on either side of said span element thereby providing the bridge formed by the span element for providing a salvaging component in lateral femoral cortex blowout conditions in ACL or PCL reconstruction.

Typically, said button comprises, two holes, equidistant from the centre, on either side, of said span element, said two holes being used to pass a suture loop which provides a support loop, on either side, for the ligament graft.

Typically, said button comprises, two pairs of holes, equidistant respectively from the centre, on either side, of said span element, said two pairs of holes being used to pass a suture loop, on either side, which provides a pair of support loops for a pair of ligament grafts respectively.

In another embodiment, said holes include a transverse diametric rod member.

For the button of this invention, the ‘bridge principle’ is adapted to be used, wherein the span of the bridge is typically supported by means of support at extreme ends i.e. without any support in the centre or across the span of the bridge. The “bridge” principle basically relies on the distance or the “Bridge” provided in between two pre-defined holes of an implant or in between the two strands of a single loop or in between the two strands of two different loops of ethibond suture, mersilene tape, polyester or any suitable strong material.

The bridge principle may be defined as the method of stabilizing a cortical button like the bridge button, of this invention, across a lateral cortical hole of a femoral tunnel, particularly the blowout hole, by spacing the loops of a suture for holding the graft(s) at a distance nearly equal to the tunnel diameter so as to prevent side to side movement of the button and so as to provide adequate cover of the button on either side of the tunnel; or by providing a collar almost equaling the tunnel diameter onto the undersurface of the button to engage securely inside the tunnel.

The “bridge” principle, which defines the incorporation of a bridge, basically relies on the distance or the “Bridge” provided in between the holes of the implant or in between the two strands of a single loop or in between the two strands of two different loops of sutures. The sutures may be ethibond suture, mersilene tape, polyester or any suitable strong material.

This bridge is of critical importance to effectively space the suture loop inside the blowout tunnel; e.g. in a 10 mm cortical blowout an 8 mm bridge with the suture loops can effectively fill the tunnel internally by the suture loops leaving no space for side to side movement of the implant and effects rigid, secure, reliable and reproducible femoral cortical fixation. This also eliminates the risk of dislodgement or loss of fixation completely.

### BRIEF DESCRIPTION OF THE ACCOMPANYING DRAWINGS

The invention will now be described in relation to the accompanying drawings, in which:

FIGS. 1a and 1b illustrate a schematic of the lateral condyle of the femur bone with AM tunnel and PL tunnel according to different surgical procedures;

FIG. 2 illustrates a schematic of the AM tunnel and PL tunnel in the tibia;

FIG. 3 illustrates a schematic of the button used for double bundle reconstruction surgery with the sutures therein and a spacer element in between the bundles;

FIGS. 4 and 5a illustrate a schematic of the button used for single bundle reconstruction surgery and in case of lateral cortex blowout;

FIG. 5b illustrates a schematic of the button used for double button reconstruction surgery and in case of lateral cortex blowout;

FIGS. 6a, 6b, 6c, 6d, 6e, 6f, 6g, 6h, and 6i illustrate schematics of various designs of buttons used during surgery;

FIGS. 7, 8, and 9 illustrate bridge buttons along with effective bridge span defined by the holes and suture loops used;

FIGS. 10a, 10b, 10c, 11a, 11b, 11c, and 11d illustrate various views of a new anchorage device or button for ligament reconstruction;

FIGS. 12a, 12b, 12c, and 12d represent additional designs of bridge button for ligament reconstruction;

FIG. 13a illustrates a graphical view which compares EB, BB, and XTB for a 10 mm tunnel in terms of percentage of success;

FIG. 13b illustrates a graphical view which compares EB, BB, and XTB in terms of percentage of success;

FIG. 13c illustrates a graphical view which compares EB, BB, and XTB for a 10 mm tunnel in terms of percentage of success; and

FIG. 13d illustrates a graphical view which compares EB, BB, and XTB in terms of percentage of success.

### DETAILED DESCRIPTION OF THE ACCOMPANYING DRAWINGS

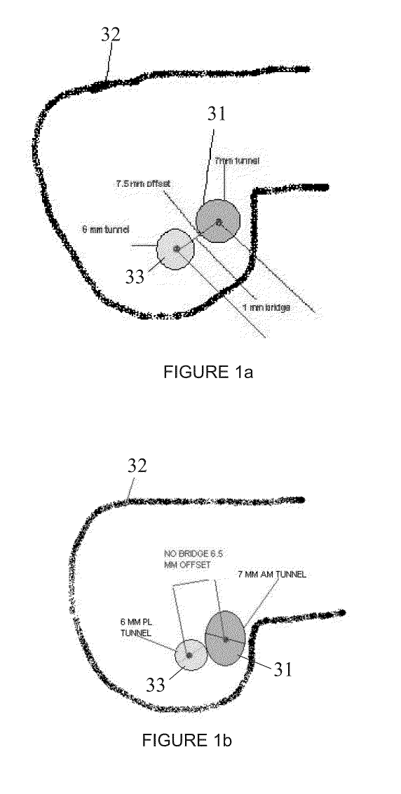

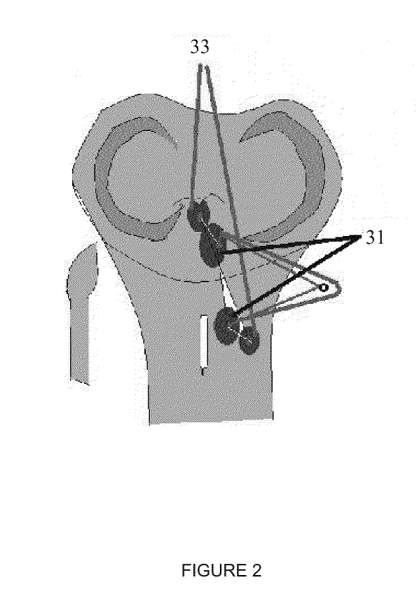

FIGS. 1a and 1b illustrate a schematic of the lateral condyle (32) of a femur bone with AM tunnel (31) and PL tunnel (33) according to different surgical procedures. FIG. 2 illustrates a schematic of the AM tunnel (31) and PL tunnel (33) in the tibia. Reference numeral 35 refers to tuberal tuberosity.

FIG. 2 illustrate a schematic of various AM tunnel and PL tunnel in the tibia.

According to this invention, there is provided a button (100) formed by an elongate body of pre-defined dimensions. The button body has pre-defined dimensions so that the button is adapted to sit across a cavity in a bone.

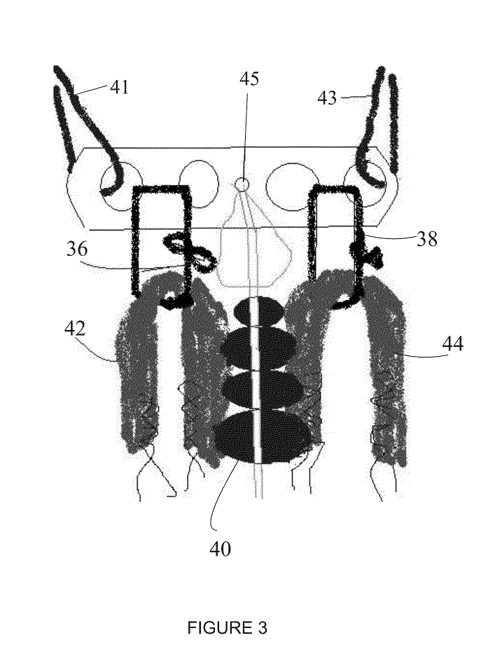

FIG. 3 illustrates a schematic of the button used for double bundle reconstruction surgery with the sutures therein and a spacer element (40) in between the bundles (42, 44). Reference numeral 42 refers to AM bundle. Reference numeral 44 refers to PL bundle. Reference numeral 41 refers to pulling sutures. Reference numeral 43 refers to flipping sutures. Hole (45) for interference screw thread is centrally located.

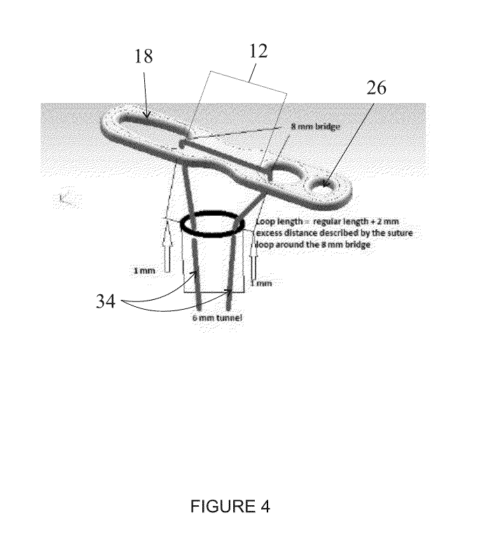

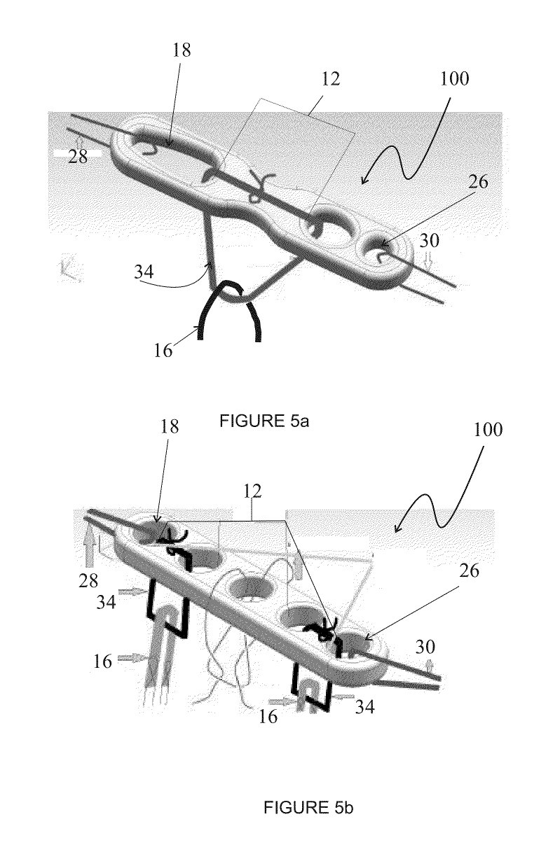

FIGS. 4 and 5a illustrate a schematic of the button used for single bundle reconstruction surgery and in case of lateral cortex blowout.

FIG. 5b illustrates a schematic of the button used for double button reconstruction surgery and in case of lateral cortex blowout.

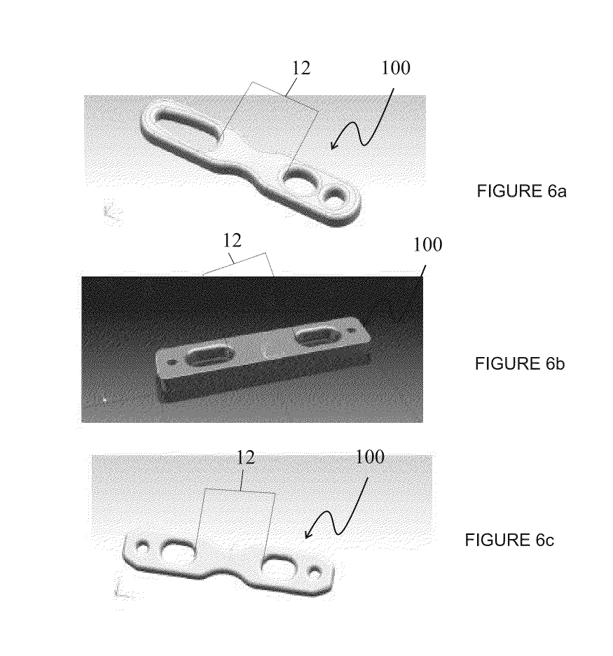

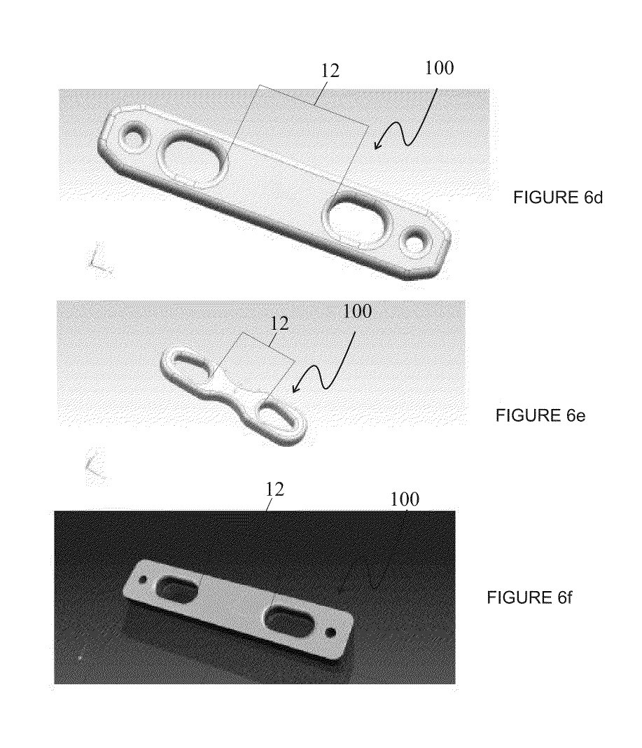

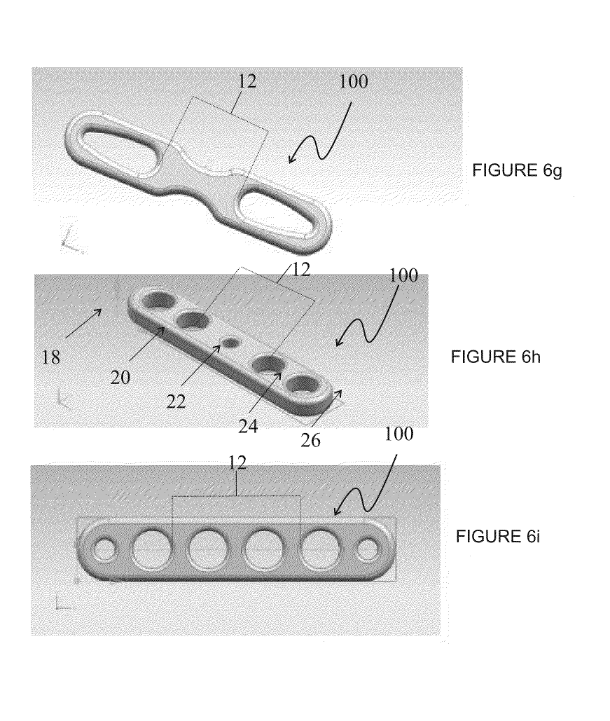

FIGS. 6a, 6b, 6c, 6d, 6e, 6f, 6g, 6h, and 6i illustrate schematics of various designs of buttons used during surgery.

FIG. 6a refers to Bridge Button Single Tunnel (ST) large. FIG. 6e refers to Bridge Button Single Tunnel (ST) small.

In accordance with an embodiment of this invention, there is provided a solid member (12), located substantially at the centre of said elongate body of said button, adapted to define a bridge portion. The span of the solid member is defined in accordance with the cavity (14) it is adapted to sit across.

The span of the solid member may be solid or may include a plurality of holes for various purposes apart other than passing sutures for holding the graft (16).

In accordance with another embodiment of this invention, there is provided a plurality of holes (18, 20, 22, 24, 26) at pre-determined locations, beyond said bridge, adapted to allow passing of sutures for holding said graft, sutures for handling said button, sutures for handling an insert and the like purposes. The holes may be selected in various combinations.

A first laterally located hole (18) at a first longitudinal edge of the button may be used for passing sutures which act as the pulling suture (28). This pulling suture aids in pulling the button along with graft through a formed tunnel. Typically, the button is passed through the tunnel in an operative vertical position along its longitudinal axis.

A second laterally located hole (26) at a second longitudinal edge (longitudinal diametrically opposite to said first longitudinal edge) of the button may be used for passing sutured which act as the flipping suture (30). This flipping suture is typically engaged, by the surgeon, once the button has been pulled out of the tunnel on the lateral side of the bone. It aids in flipping (configuring) button from its operative vertical position (for pulling purposes) to its operative horizontal position wherein it can rest upon the cavity, transversely.

Ideally, the bridge element sits across the cavity with the lateral exterior-most holes placed outside the cavity and resting upon the solid portion of the bone.

In a preferred embodiment and method, the bridge is marginally lesser than the cavity. The margin which is the differential incorporates the diameter of the sutures on either end. Thus the sutures pass through the inner edge of the holes and are plugged in between said inner edge of the hole and the wall of the tunnel. This provides a secure fit of the button onto the cavity.

In a preferred embodiment, the holes are placed on either side of said solid bridge element whereby providing the bridge formed by the solid element for providing a salvaging component in lateral femoral cortex blowout conditions in ACL or PCL reconstruction.

There is provided a technique of Bridge Button in the novel bicortical tunnel “no socket” ACL or PCL reconstruction surgery.

In a single bundle loop technique, two holes, equidistant from the centre, on either side, are used to pass a suture loop (34) which provides a support loop for the ligament graft. (as shown in FIG. 5)

In a double loop technique, two pairs of holes, equidistant respectively from the centre, on either side, are used to pass a suture loop (36, 38) on either side which provides a pair of support loops for a pair of ligament grafts respectively. (as shown in FIGS. 3 and 5b)

The current invention includes more than one preferred design of button. There may be provided two or more holes strategically spaced along the body based upon the bridge principle, i.e. on either side of the solid element. Alternatively, holes may be provided through the solid element, too. But, the pulling suture and the flipping suture are passed through the exterior-most holes. The intermediate holes aid in passage of the sutures which hold the graft in the tunnel.

The implant provides strong cortical fixation at both the femoral and tibial ends, separates the graft into two distinct bundles, allows individual tensioning and fixation of the two bundles in different flexion angles and can be combined with aperture fixation like bio screw or the special insert or cage.

In short, it is a versatile implant with multiple applications and is an extremely helpful salvage implant in case of femoral tunnel blowout in single bundle ACL or PCL reconstructions or in inadvertent tunnel communication in double bundle anatomic ACL or PCL reconstruction.

The “bridge” principle, which defines the incorporation of a bridge, basically relies on the distance or the “Bridge” provided in between the holes of the implant or in between the two strands of a single loop or in between the two strands of two different loops of sutures. The sutures may be ethibond suture, mersilene tape, polyester or any suitable strong material.

This bridge is of critical importance to effectively space the suture loop inside the blowout tunnel; e.g. in a 10 mm cortical blowout, an 8 mm bridge with the suture loops can effectively fill the tunnel internally by the suture loops leaving no space for side to side movement of the implant and effects rigid, secure, reliable and reproducible femoral cortical fixation. This also eliminates the risk of dislodgement or loss of fixation completely.

In one exemplary embodiment of the design, the button is about 25 mm in length elongated in shape, about 5 mm in breadth and about 2 mm in thickness. It is, preferably, made of Titanium.

In another preferred exemplary embodiment, there may be provided three holes: (a) one large hole on one end and two holes on the other end. All the holes in the implant are filleted and extremely smooth from inside to prevent suture loop attrition or cutout.

The bridge or distance between the large hole and the side hole is typically 8 mm in accordance with the bridge principle. This bridge is of critical importance to space the suture loop in the blowout tunnel (e.g. 10 mm blowout) so that the implant does not move side to side and does not get dislodged back into the tunnel with cyclic loading or motion of the knee after tibial fixation in the postoperative period. This implant can be safely used for tunnel blowouts from 6 mm to 10 mm.

The small bridge button ST is used for about 6 to 10 mm blowouts and the larger one used for about 11 to 13 mm blowouts.

Alternatively, the button of the current invention may have only two equal pear shaped holes or may have four or more holes. For larger than 10 mm blowouts, the intervening bridge may be more than 8 mm-generally 2 mm less than the tunnel diameter, e.g. for 12 mm blowout the bridge may be 10 mm which along with the 2 mm suture loop can fill the internal diameter of the tunnel thus eliminating side to side movement of the button and providing absolutely secure fixation. The length of 25 mm can cater to blowouts from 6 mm to 17 mm.

The button is designed to be used with 3 or 4 suture loops. However, it may be compatible for use with a continuous polyester loop technology like Smith & Nephew Endobutton CL® or with a self locking adjusting suture loop technology like the TightRope® by Arthrex® or any other suitable strong material like fibrewire or the like. These suture loops are adapted to suspend the graft and are passed through the pear shaped hole and its adjacent circular hole and a desired length of strong loop is created over which the four-strand hamstring graft will be draped.

The leading strong suture or fibre wire is passed through the end of the pear shaped hole and this is the leading (pulling) end of the button. The flipping suture is passed through the last hole in the implant and this is the trailing (flipping) end of the button.

The button can be used for single bundle anatomic ACL OR PCL reconstruction where instead of creating a stepped socket a single bicortical tunnel of the desired graft diameter is created in the anatomical insertion site of the ACL in the lateral femoral condyle and the graft attached to the button with suture loops is passed and locked onto the lateral femoral cortex. This can save time during surgery.

Typically, in a 10 mm tunnel blowout hole the security of fixation of the Bridge button ST was checked by the ability of the implant to resist all and any attempts to dislodge it into the tunnel whilst holding the central loop of sutures passed through the central holes down the femoral tunnel securely. The Bridge button withstood this challenge test, successfully.

Similarly, this same button with same bridge element and dimension may be used for Anatomic double bundle ACL or PCL reconstruction based on the same bridge principle and labelled as “Bridge Button DT” where DT stands for double tunnel double bundle ACL or PCL reconstruction.

The Bridge button DT is, preferably, about 25 mm in length, about 5 mm in breadth and about 2 mm in thickness and is made of Titanium. It has rounded ends and generally smooth on all surfaces. It may typically have 5 holes; all of which are filleted with smooth inside surfaces to prevent suture attrition or cutout. All 5 holes are, preferably, about 3 mm in diameter.

The outer two holes in the Bridge button DT are used to create two separate loops of sutures of desired length depending on the tunnel length and the desired graft insert. These two loops will carry the two separate graft bundles and replace the two bundles of the anterior cruciate ligament. Alternatively, the loops may be compatible for use with the continuous polyester technology or the self locking technology available in the market.

According to another exemplary embodiment, the design of the Bridge button DT allows a bridge of about 7 mm between the two loops and hence between the two bundles and maintains the space between these two bundles effectively to act as anteromedial and posterolateral bundles just as in a normal native ACL.

According to yet another exemplary embodiment, the design of the Bridge button DT further allows a bridge of 16 mm between the outer borders of the two loops and thus can effectively bridge a combihole tunnel of 17 mm providing adequate cover of 4 mm on the lateral femoral cortex on either side of the tunnel.

The central hole in the Bridge button DT is for passing a suture or fibre wire between the two bundles and to be pulled out through the anteromedial portal. This suture will guide the passage of a Bioscrew over a cannulated screwdriver to be placed in between the two bundles providing aperture fixation as well as forcing the graft against the tunnel walls for better healing; at the same time ensuring the two bundle anatomy of the reconstructed ACL or PCL.

An implant designed for use in lateral cortex blowout during ACL or PCL reconstruction surgery using the “bridge” principle given labelled as “Bridge Button ST” where ST stands for Single Tunnel ACL or PCL reconstruction.

The Bridge button ST is, typically, available in two sizes:
(a) Bridge button ST Small-20 mm length×5 mm width×2 mm thickness for tunnel blowouts from 6 mm to 10 mm and
(b) Bridge button ST Large-25 mm length×5 mm width×2 mm thickness for tunnel blowouts from 11 mm to 13 mm.

The bridge or distance between the pear shaped hole (a) and the side hole (b) is 8 mm in accordance with the bridge principle in claim 1. This bridge is of critical importance to space the suture loop in the blowout tunnel (e.g. 10 mm blowout) so that the implant does not move side to side

The 8 mm bridge along with the suture loops provide a total bridge of at least 8+2=10 mm.

Following table 1 summarises the play allowed in different tunnel diameters by the bridge button ST and the cover provided by the buttons:

TABLE 1

| | | | TOTAL | PLAY |
|---|---|---|---|---|
| | | | COVER | WITH |
| BRIDGE | TUNNEL | | ON BOTH | SUTURE |
| BUTTON ST | DIAMETER | PLAY | SIDES | LOOPS |
| SMALL- 20 MM | 6 MM | NIL | 7.0 MM | NIL |
| LARGE 25 MM | 6 MM | NIL | 9.5 MM | NIL |
| SMALL- 20 MM | 7 MM | NIL | 6.5 MM | NIL |
| LARGE 25 MM | 7 MM | NIL | 9.0 MM | NIL |
| SMALL- 20 MM | 8 MM | NIL | 6.0 MM | NIL |
| LARGE 25 MM | 8 MM | NIL | 8.5 MM | NIL |
| SMALL- 20 MM | 9 MM | 1 MM | 5.5 MM | NIL |
| LARGE 25 MM | 9 MM | 1 MM | 8.0 MM | NIL |
| SMALL- 20 MM | 10 MM | 2 MM | 5.0 MM | NIL |
| LARGE 25 MM | 10 MM | 2 MM | 7.5 MM | NIL |
| SMALL- 20 MM | 11 MM | 3 MM | 4.5 MM *NR | 1 MM |
| LARGE 25 MM | 11 MM | 3 MM | 7.0 MM | 1 MM |
| SMALL- 20 MM | 12 MM | 4 MM | 4.0 MM *NR | 2 MM |
| LARGE 25 MM | 12 MM | 4 MM | 6.5 MM | 2 MM |
| SMALL- 20 MM | 13 MM | 5 MM | 3.5 MM *NR | 3 MM |
| LARGE 25 MM | 13 MM | 5 MM | 6.0 MM | 3 MM |

NR = NOT RECOMMENDED

An implant designed for Anatomic double bundle ACL or PCL reconstruction based on the same bridge principle and labelled as “Bridge Button DT” where DT stands for double tunnel double bundle ACL or PCL reconstruction.

The Bridge button DT is typically about 25 mm in length, 5 mm in breadth and 2 mm in thickness and is made of highest quality Titanium.

It has rounded ends and generally smooth on all surfaces. It has preferably 5 holes all of which are filleted with smooth inside surfaces to prevent suture attrition or cutout. All 5 holes are about (preferably) 3 mm in diameter.

Alternatively, a spacer element (bioabsorbable cage) with multiple holes shaped to fit in between the graft bundles and packed with the bone grafts recovered from drilling the tunnels can be used for both the tibia and the femur to act as a biologic spacer.

This implant or the Bridge Button provides a very simplified technique of the complex double bundle ACL reconstruction which many single bundle surgeons may find easy to perform and also provides a backup salvage implant in case of complications like tunnel communication or blowout.

The versatility of the implant makes it conducive to be included in the inventory of any orthopedic surgeon treating ACL or PCL tear.

The Bridge button is not only an alternative implant but an independent salvage implant catering to blowouts from 6 mm to 17 mm.

Any implant claiming to salvage a blowout tunnel must follow the bridge principle and if it does not do so it is doomed for failure as assessed by the “challenge test”.

The AM bundle is posterior on the femoral condyle to the PL bundle in knee flexion. With the Bridge button technique it is possible to rotate the bundles to orient them anatomically to their respective positions which may not be possible with any other technique or implants; hence it is the only and unique method to restore the two bundle anatomy without the difficulties in conventional double bundle ACL reconstruction surgery.

With the advent of anatomic ACL reconstruction the femoral tunnel tends to be short at times critically short that is less than 25 mm short where Endobutton CL® with smallest loop of 15 mm is impossible to use. The free hand technique with manual loop knotting or the Arthrex TightRope® technology is the only way to address tunnels smaller than 25 mm apart from the Endobutton Direct® from Smith & Nephew® which cannot provide a bridge to separate the two bundles and can be used only for single bundle ACL reconstruction.

The smaller the tunnel and hence the loop the greater the difficulty in maneuvering the Bridge button out the femoral tunnel. The TightRope® technology can permit a longer loop for locking of the Bridge button and allow individual passage of the two bundles with more ease.

Alternatively, the tendons can be separated with a Bioscrew to allow separation of the two bundles or a RCI Screw.

The PCL reconstruction is done on the same principles using the bridge button and some special instruments.

Although the “Bridge” is primarily a function of the implant design, the thickness of the suture loops adds to the effective bridge. It is possible to increase the Bridge in a 4-hole design or in a multi-hole button by passing the sutures through peripheral 2 holes instead of the routine central 2 holes.

This means that in a multi-hole button it is possible to choose the bridge as per the tunnel diameter by passing the suture loops housing the graft through either the adjacent or the distant holes.

The only link between the bridge button and the exit hole cortex interface is the suture loop or CL loop holding the graft. Therefore the only way to stabilize the button on the lateral femoral cortex and to reduce its side to side movement is by spanning the suture bridge using the bridge principle. This imparts unprecedented stability and eliminates the risk of dislodgement of the button back into the tunnel completely.

This bridge in the implant design is of critical importance to effectively space the suture loop inside the blowout tunnel; e.g. in a 10 mm cortical blowout an 8 mm bridge along with the suture loops can effectively fill the tunnel internally by the suture loops leaving no space for side to side movement of the implant and effects rigid, secure, reliable and reproducible femoral cortical fixation.

The bridge buttons are designed, preferably, to be used with 3 or 4 suture loops of simple No. 5 Ethibond suture or mersilene tape with or without ethibond loops. However, they may be compatible for use with a continuous polyester loop technology like the Endobutton CL (SMITH & NEPHEW) or with a self adjusting suture loop technology like the TightRope (ARTHREX, NAPLES) or any other suitable strong material like fibrewire or the like.

Another way to increase the bridge is to adjust the bridge in the implant design by giving it an undersurface collar of 8 mm which can engage inside the tunnel securely without relying on the suture loops. The under collar engages in the tunnel mouth and prevents side to side movement.

According to this invention, there is provided an anchorage device or button formed by an elongate body of pre-defined dimensions. The button body has pre-defined dimensions so that the button is adapted to sit across a cavity in a bone. There is provided an under-collar which is a protrusion of pre-defined height and pre-defined length in accordance with the bridge principle, said protrusion being located substantially at the centre of the anchorage device or button. There are holes provided on the protrusion as well as on the shoulders of the button. This new XTB BRIDGE with inbuilt bridge can be used with the regular endobutton CL, It exhibits no side to side movement, and offers secure locking and fixation unlike the Xtendobutton® in a 10 mm tunnel.

Yet another way to increase secure locking and fixation is to provide a recessed operative top surface on the button which is co-axial to the button.

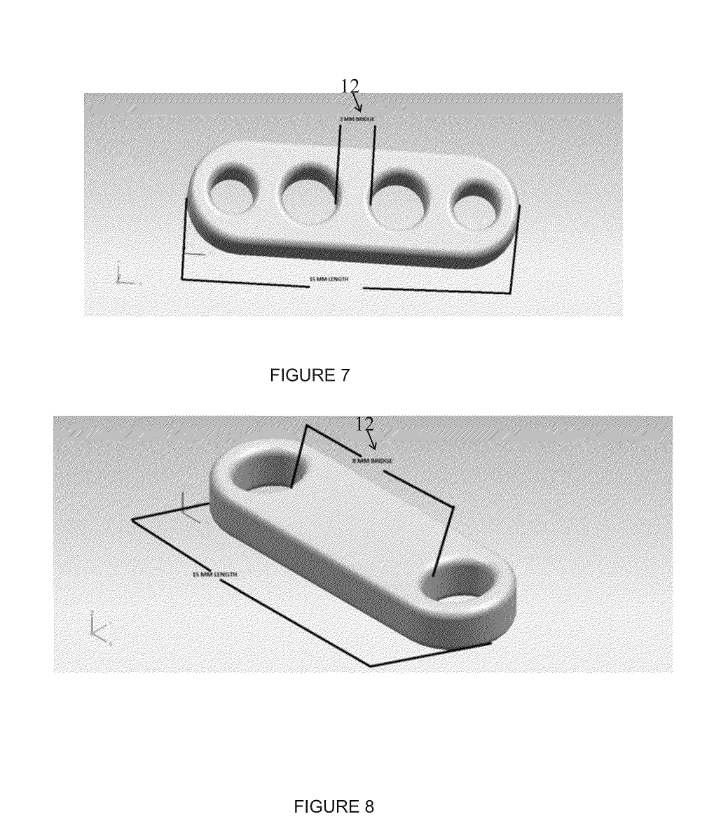

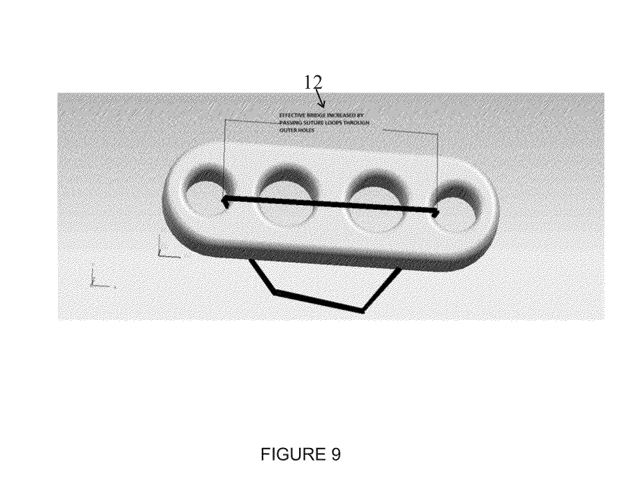

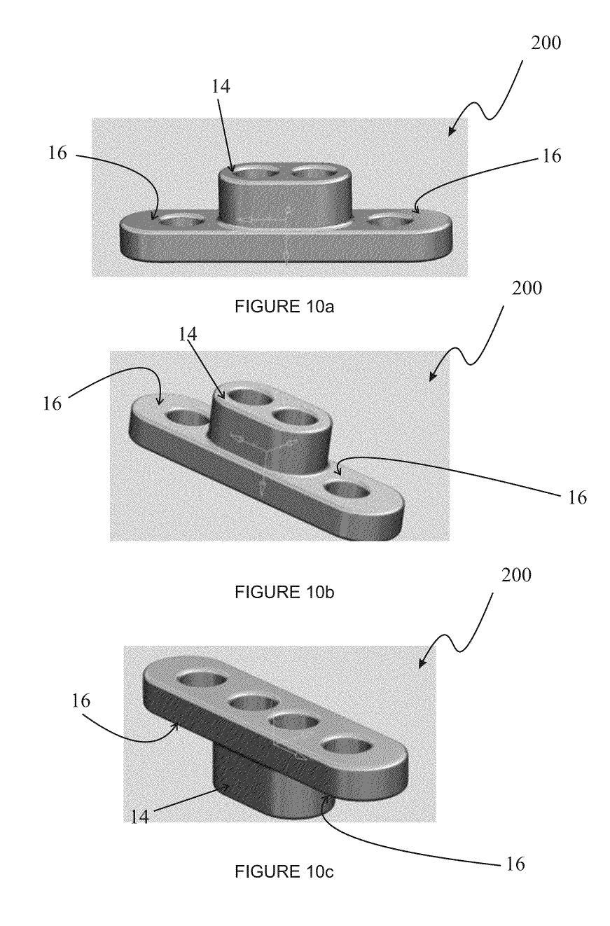

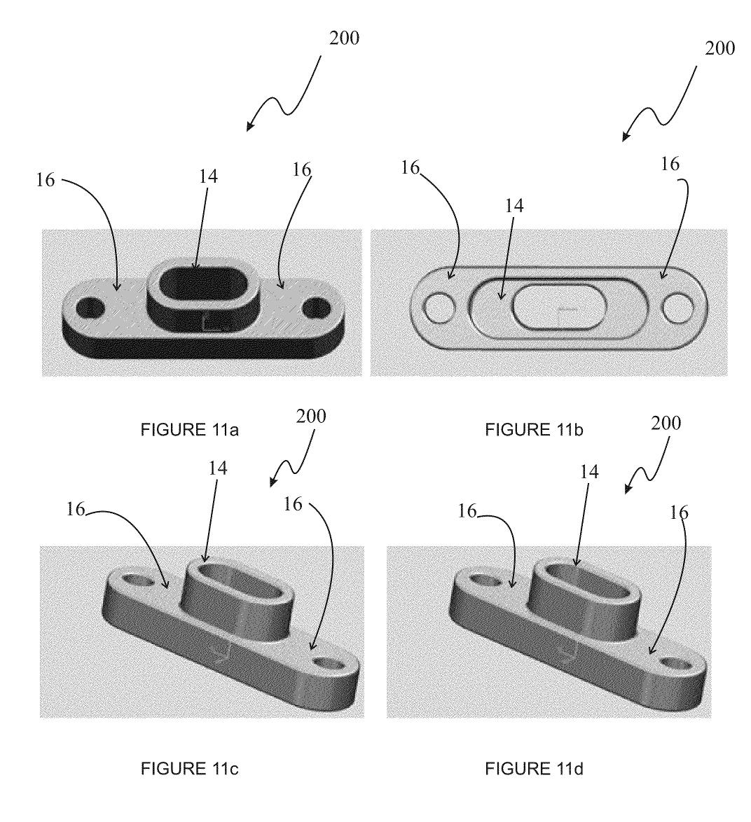

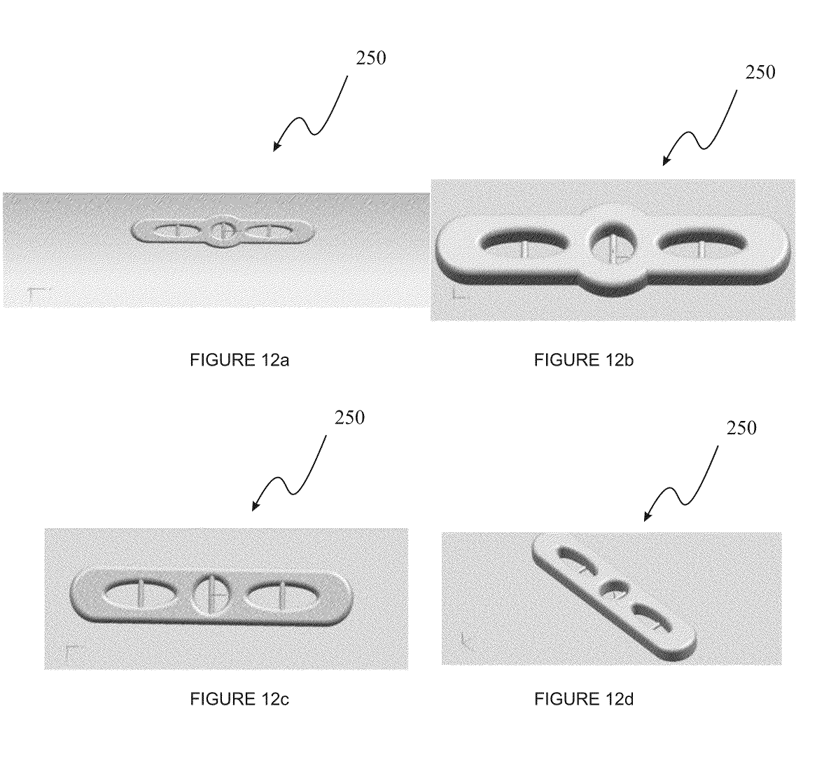

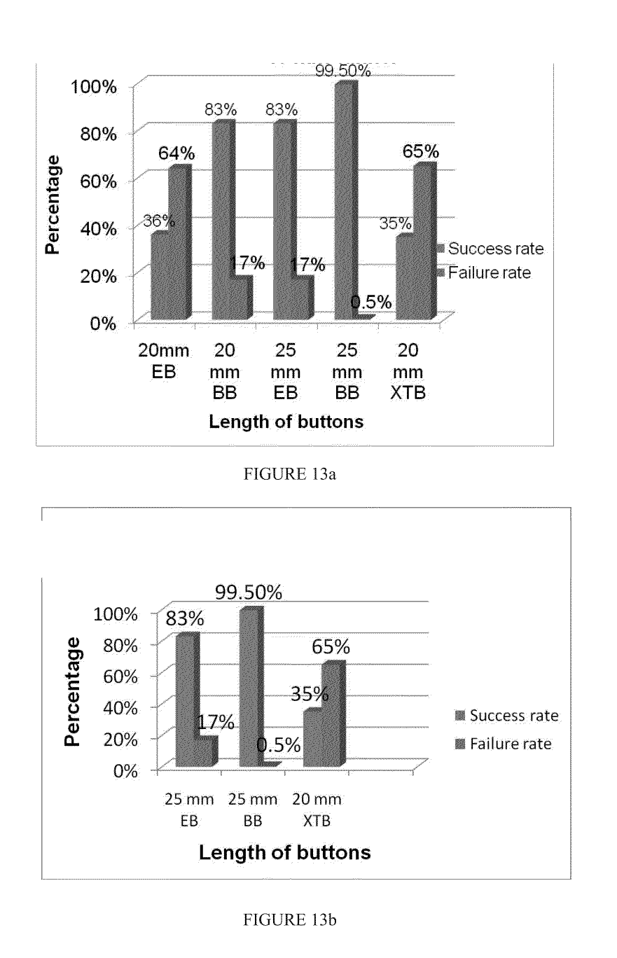

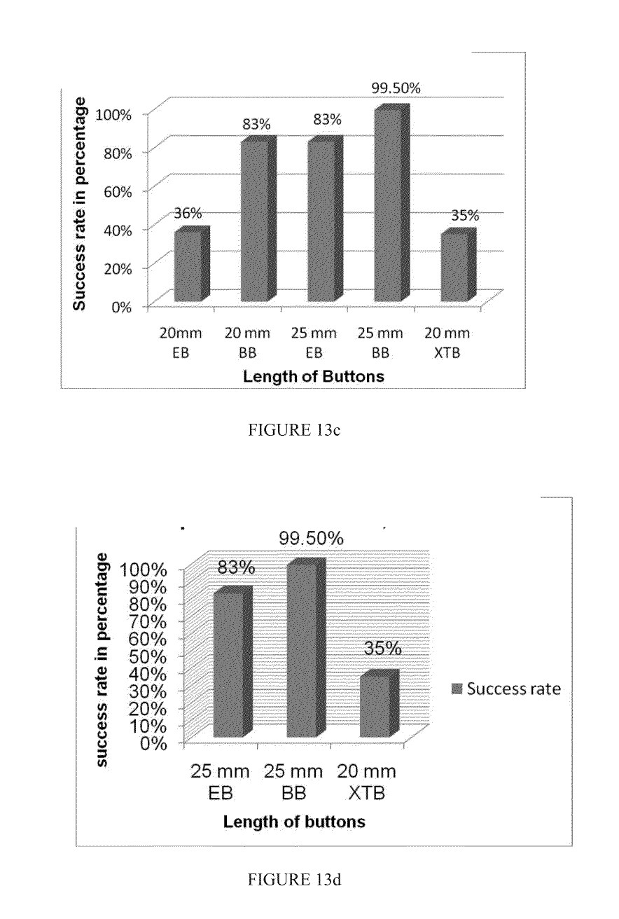

FIGS. 7, 8, and 9 illustrate bridge buttons along with effective bridge span defined by the holes and suture loops used.

Although the “Bridge” is primarily a function of the implant design, the thickness of the suture loops adds to the effective bridge. It is possible to increase the Bridge in a 4-hole design or a multi-hole button by passing the sutures through the peripheral 2 holes instead of the routine central 2 holes. This is seen in FIG. 2 of the accompanying drawings.

This means that in a multi-hole button (like the 4 hole BB or Bridge button Ultimate) it is possible to choose the bridge as per the tunnel diameter by passing the suture loops housing the graft through either the adjacent or the distant holes. The only link between the endobutton or bridge button and the exit hole cortex interface is the suture loop or CL loop holding the graft. Therefore, the only way to stabilize the button on the lateral femoral cortex and to reduce its side to side movement is by spanning the suture bridge using the bridge principle. This imparts unprecedented stability and eliminates the risk of dislodgement of the button back into the tunnel completely. This bridge in the implant design is of critical importance to effectively space the suture loop inside the blowout tunnel; e.g. in a 10 mm cortical blowout an 8 mm bridge along with the suture loops can effectively fill the tunnel internally by the suture loops leaving no space for side to side movement of the implant and effects rigid, secure, reliable and reproducible femoral cortical fixation. The Bridge buttons are designed to be used with 3 or 4 suture loops of simple No. 5 Ethibond suture or mersilene tape with or without ethibond loops. However, they may be compatible for use with a continuous polyester loop technology like the Endobutton CL (SMITH & NEPHEW) or with a self locking suture loop technology like the TightRope (ARTHREX, NAPLES) or any other suitable strong material like fibrewire or the like.

Another way to increase the bridge is to adjust the bridge in the implant design by giving it an undersurface collar of 8 mm which can engage inside the tunnel securely without relying on the suture loops. In this case the implant may have holes separated by 2 mm or lesser bridge:

FIGS. 3a, 3b, 3c, 4a, 4b, 4c, and 4d illustrate various views of a new anchorage device or button for ligament reconstruction.

According to this invention, there is provided an anchorage device or button (200) formed by an elongate body of pre-defined dimensions. The button body has pre-defined dimensions so that the button is adapted to sit across a cavity in a bone. There is provided an under-collar (14) which is a protrusion of pre-defined height and pre-defined length in accordance with the bridge principle, said protrusion being located substantially at the centre of the anchorage device or button. There are holes provided on the protrusion as well as on the shoulders (16) of the button. This new XTB BRIDGE with inbuilt bridge can be used with the regular endobutton CL, It exhibits no side to side movement, and offers secure locking and fixation unlike the Xtendobutton in a 10 mm tunnel.

The under collar engages in the tunnel mouth (of hole made in bone) and prevents side to side movement.

SIDE TO SIDE MOVEMENT=(tunnel diameter−the bridge in the implant design+suture diameter)/2:

For the purposes of this specification, ‘cover’ may be defined as the amount of bone covered by the implant after fixation and depends on the bridge provided in the implant design and the length of the implant. (In practice, the thickness of the suture loops adds to this bridge in the implant design.)

When the bridge equals the tunnel diameter no side to side movement is possible and the cover is equal to the Length of the implant−the tunnel diameter/2 and equal on either side of the tunnel.

With bridge smaller than the tunnel diameter the button obviously tends to be pulled on one side resulting in more cover on one side and suboptimal cover on the other.

As the exit hole is usually oval and longer in supero-inferior axis than the transverse axis by about 2 mm the cover required in actual practice needs to be more than this formula.

Simple calculation of cover (Length of the implant−the tunnel diameter/2) gives an immediate rough idea if an implant can provide fixation. e.g. a 12 mm implant by no stretch of imagination can be expected to provide fixation in a 13 mm tunnel; whereas in a tunnel of 6 mm one can easily gauge that the expected cover is 12−6=6 mm/2 i.e. 3 mm on either sides and this might provide fixation.

Therefore “effective cover” is dependent on the “Bridge” of the implant and the side to side movement permitted by this bridge. It may be defined as the cover provided by the button in reality as per the tunnel diameter, bridge length and overall length of the implant.

(As the exit hole is usually oval and longer in supero-inferior axis than the transverse axis by about 2 mm the cover required in actual practice needs to be more than this simple formula).

Simple calculation of cover (Length of the implant−the tunnel diameter/2) gives an immediate rough idea if an implant can provide fixation. e.g. a 12 mm implant by no stretch of imagination can be expected to provide fixation in a 13 mm tunnel; whereas in a tunnel of 6 mm one can easily gauge that the expected cover is 12−6=6 mm/2 i.e. 3 mm on either sides and this might provide fixation.

With a 15 mm button with a bridge of 2 mm in a tunnel diameter of 8 mm, the side to side movement permitted will be:

8 (tunnel diameter)−2 (bridge)/2 i.e. (8−2/2)−3 mm

Therefore the “Effective cover” is 3.5 (cover)-3.0 (side to side movement)=0.5 mm only.

As the bridge of 2 mm in the implant design permits 3 mm of side to side movement the effective cover is only 0.5 mm although the so called expected cover is 3.5 mm and therefore the 15 mm endobutton is expected to fail in 8 mm tunnel in providing secure fixation.

Whereas, a 15 mm button with a bridge of 8 mm does not permit any side to side movement in an 8 mm tunnel. Therefore the cover of 15−8/2=7/2 mm i.e. 3.5 mm on each side is sufficient to provide secure fixation which the button with 2 mm bridge cannot provide although the length may be the same.

The above mentioned discussion clearly highlights the importance of the bridge principle in effecting stability to the button whichever design it might have; it also clearly proves that length although important to provide cover does not necessarily ensure secure fixation as absence of adequate bridge equaling the tunnel diameter adds another dimension to the button tunnel interface i.e. the side to side movement permitted which can compromise fixation by affecting the cover even if the implant has a length longer than the tunnel diameter.

The technical advancement lies in the fact that this fixture (bridge button) is a single assembly which provides a firm placement across a cavity of the bone without any scope for dislodgement or failure. The ‘bridge principle’ is adapted to be used, wherein the span of the bridge is typically supported by means of support at extreme ends i.e. without any support in the centre or across the span of the bridge. This is a cost-effective solution, even in cases of blowout. The advantage of this anchorage device lies in the fact that the solid element (i.e. bridge) transverses the cavity of the bone, and deftly and firmly sits across the cavity without any chance of inadvertent displacement (either during surgery or post-surgery) and also guarantees it not falling into the cavity, itself. Fillets in the holes ensure smooth margins and do not allow suture attrition or cutout. The same button can be used for ACL reconstruction with single bundle technique ranging from 6 mm to 17 mm graft diameter or blowout without having to ream a socket, thus saving time. It is useful for outside in tunnel drilling techniques. It is useful in children where growth plate avoidance may dictate outside in drilling. Single member assembly avoids the possibility of disengagement. It has a low profile; yet strong, secure and reliable implant. The locking is completely secure and reproducible. One implant can be used for primary ACL reconstruction or revision or blowouts or for elective single tunnel no socket technique as well as for double bundle ACL or PCL reconstruction. Same implant can be used with Closed Continuous polyester loop technology. Same implant can be used with the Tight Rope technology. This increases modularity. Can also be used for other indications like PCL reconstruction, MCL reconstruction etc. It avoids use of two implants in double bundle reconstructions. It is understood that various designs with varying bridge lengths and number of holes can be used with advantage for different indications. The option of aperture fixation with interference screw between the two bundles is possible. It can restore the anatomy of ACL more closely by allowing rotation of the two bundles.

Generally, in case of lateral cortex blowout a suture disc or post can be used to fix the graft on the lateral femur but this requires additional incision on the lateral thigh, is time consuming and adds to the pain and scar. With the Bridge button no lateral incision is required and the button can be passed through the tibial and femoral tunnels itself.

One implant does it all from single tunnel single bundle to double tunnel double bundle surgery in addition to providing salvage backup in cases of cortical blowouts

According to a non-limiting exemplary embodiment, a study was done on an anatomically correct Swiss made femur model “Synbone” 2230. Serial tunnel blowouts were made after inserting the guide wire in the anatomic centre of the ACL footprint from 5 mm to 10 mm and each button was tried for correct placement and locking 20 times each for 5 to 9 mm tunnels (n=20) and 100 times for 10 mm tunnel (n=100). Femur model was used instead of knee model to eliminate the possibility of the suture loop passing through the tibial tunnel providing a pre-determined direction of pull forcing the button to be pulled in an inferior and anterior direction and assisting in proper secure placement of the buttons. A single loop of no. 2 ethibond was passed through the central 2 holes of all the buttons and marking was made on the thread at the tunnel length+the length of the implant to mark the flipping distance. Single loop was preferred to avoid increase in the effective “Bridge” due to the thickness of the suture and to eliminate the effects of the same. In other words, the bridge in the implant was the only goal of the study. No graft or artificial substitute was attached to the loop to prevent the contribution made by the graft to centralize the loop and to assist in the proper placement of the buttons, and a standard uniform weight of 100 gms was attached to the ethibond loop directly to provide traction to the suture loop. Thus, all possible factors that could contribute to the proper placement of the buttons were eliminated and the only factor which was being tested in the proper placement was the implant design i.e. its length and the “Bridge” which was the distance between the two central holes in the implant housing the suture loop meant for securing the graft. Prototypes of following buttons were made on Rapid prototyping machine Stratasys FDM 900 mc USA initially in ABS-acrylonitryle butadiene styrene and then in stainless steel on EOS INT M 270 machine Germany and compared statistically. A ‘15 mm long×5 mm wide×2 mm thick’ with 2 mm bridge (labelled as 15 mm BB-2) was compared with ‘15×5×2 mm’ Bridge button with 8 mm bridge (15 mm BB-8). I.e. 15 mm BB-2 versus 15 mm BB-8.

Further, a ‘20 mm long×5 mm wide×2 mm thick’ button with 2 mm bridge (20 mm BB-2) was compared with ‘20×5×2 mm’ Bridge button with 8 mm bridge (20 mm BB-8). I.e. 20 mm BB-2 versus 20 mm BB-8.

Still further, a ‘25 mm long×5 mm wide×2 mm thick button’ with 2 mm bridge (25 mm BB-2) was compared with ‘25×5×2 mm’ Bridge button with 8 mm bridge (25 mm BB-8). I.e. 25 mm BB-2 mm versus 25 mm BB-8 mm

All of the above compared with the Xtendobutton® (XTB). The XTB cannot function on its own; it must be coupled with the regular EB or the EB CL extending the overall length of the implant to 20 mm but utilizing the original “bridge” of the small endobutton: i.e. (1.22 mm approximately). It is a two member assembly but displays the potential for excessive side to side movement in a 10 mm tunnel blowout with inadequate cover on one side or the other mainly due to lack of the bridge principle similar to the 20 mm BB-2.

The 3 buttons, mentioned above, were made in 15, 20 and 25 mm lengths and all had the same width of 5 mm and thickness of 2 mm. Each of the three buttons was made in two bridge lengths viz. 2 mm and 8 mm.

The implants were passed serially through the blowout holes 20 times for tunnels from 5 to 9 mm and 100 times for 10 mm blowout hole. The buttons were flipped and locked at the appropriate flipping distance mark for each button. The placement of the button in terms of the cover, side to side movement, security of fixation and the response to the challenge test was recorded.

In the femur bone model study with 20 attempts at locking the results were as follows:
1. In the 5 and 6 mm tunnel (n=20), there was almost 100% success rate for all buttons including the 15 mm BB-2, 15 mm BB-8, 20 mm BB-2, 20 mm BB-8, 25 mm BB-2, 25 mm BB-8 and the Xtendobutton®.
2. In the 7 mm tunnel (n=20), all the buttons had almost 100% success rate except the 15 mm BB-2 which had 25% failure rate.
3. In the 8 mm tunnel (n=20), all the buttons had almost 100% success rate except the 15 mm BB-2 which had 65% failure rate.
4. In the 9 mm tunnel (n=20), the results were as follows:
   15 mm BB-2=100% failure rate . . . 15 mm BB-8=15% failure rate
   20 mm BB-2=50% failure rate . . . 20 mm BB-8=0% failure rate
   25 mm BB-2=0% failure rate 25 mm BB-8=0% failure rate
   Xtendobutton=0% failure rate=9%
5. In the 10 mm tunnel (n=100), the results are shown in following table no 2.

TABLE NO. 2

| | Sample | Success | Failure | Z | |
|---|---|---|---|---|---|
| Button length | size | rate | rate | value | P value |
| 15 mm BB | 100 | 28% | 72% | 7.75 | P < |
| | | | | | 0.0005*** |
| 15 mm BB | 100 | 76% | 24% | BB is superior | |
| | | | | than EB for 15 mm | |
| | | | | length of | |
| | | | | button | |
| 20 mm EB | 100 | 36% | 64% | 7.71 | P < |
| | | | | | 0.0005*** |
| 20 mm BB | 100 | 83% | 17% | BB is superior | |
| | | | | than EB for 20 mm | |
| | | | | length of | |
| | | | | button | |
| 25 mm EB | 100 | 83% | 17% | 4.325 | P < |
| | | | | | 0.0005*** |
| 25 mm BB | 100 | 99.50% | 0.50% | BB is superior | |
| | | | | than EB for 25 mm | |
| | | | | length of | |
| | | | | button | |
| 20 mm BB | 100 | 83% | 17% | 7.91 | P < |
| | | | | | 0.0005*** |
| 20 mm XTB | 100 | 35% | 65% | 20 mm BB is | |
| | | | | superior than 20 mm | |
| | | | | XTB | |
| 25 mm BB | 100 | 99.50% | 0.50% | 13.4 | P < |
| | | | | | 0.0005*** |

| | | | | |
|---|---|---|---|---|
| 20 mm XTB | 100 | 35% | 65% | 25 mm BB is |
| | | | | highly superior |
| | | | | than 20 mm XTB |

(***very highly significant)

Another study was done on an anatomically correct articulated knee model with ligaments. The ACL and PCL were excised and the patella and patellar tendon were removed to provide a clear view of the interior of the knee joint. The tibial tunnel was made in the anatomic centre of the ACL with a 10 mm reamer after passing guide wire with the help of a regular Acufex ACL Jig with tip aimer. A guide wire was passed into the anatomic centre of the femoral tunnel and outside in drilling of the femoral tunnel was done with 10 mm reamer to prevent splintering or enlargement of the tibial or femoral tunnel that can happen with the transtibial technique ( . . . ) The tunnel length was 40 mm. A single loop of 2 mm Ethibond no 2 of 20 mm was made for the 20 mm BB-2, 20 mm BB-8, 25 mm BB-2, 25 mm BB-8 and the Xtendobutton® (XTB). The 15 mm button was excluded from this study owing to its high failure rate in the previous studies in 10 mm blowout.

The XTB was tested with a 10 mm graft substitute of tape with standard weight of . . . gms applied to the suture ends to provide traction and was also tested with Ethibond no. 2 passed through the suture loop. The XTB was pulled through the tibial tunnel and the femoral tunnel 100 times and flipped followed by automatic retrieval of the XTB and locking of the XTB due to the traction force applied by the weight to the graft/ethibond suture (n=100). No significant difference was observed in the two groups indicating that the graft or substitute had little contributory role in the correct placement of the button.

Next, the 20 mm BB-8 was tested with graft substitute and compared with the XTB as both had the same length but different bridges.

This was followed by testing of the 20 mm BB-2, 25 mm BB-2, and 25 mm BB-8 buttons. On Knee model, Table No. 3 readings were observed:

TABLE NO. 3

| Button | Sample | | | | |
|---|---|---|---|---|---|
| length | size | Success rate | Failure rate | Z value | P value |
| 20 mm BB | 100 | 96% | 4% | 5.06 | P< |
| | | | | | 0.0005*** |
| 20 mm XTB | 100 | 71% | 29% | 20 mm BB is | |
| | | | | superior than | |
| | | | | 20 mm XTB | |
| 25 mm BB | 100 | 98.00% | 2% | 5.68 | P< |
| | | | | | 0.0005*** |

| | | | | |
|---|---|---|---|---|
| 20 mm XTB | 100 | 71% | 29% | 25 mm BB is |
| | | | | highly superior |
| | | | | to 20 mm XTB |

(***very highly significant)

Tests were performed 25 mm BB and 20 mm XTB on Knee Model, and comparisons were made. After performing experiments for each button, n=100 times to test secure locking of the buttons on Knee Model.

Alternative Hypothesis (H1): The 25 mm BB is superior than 20 mm XTB in surgery.

By using Z test, Difference is highly significant between the results of 25 mm BB and 20 mm XTB.

Therefore, alternative hypothesis is accepted at P<0.0005.

Conclusion: The 25 mm BB is superior than 20 mm XTB for 10 mm tunnel in terms of secure locking, cover and proper placement of the buttons on knee model at 0.0005 level of significance.

Results of the Knee Model study were as follows:
1. In a 10 mm tunnel blowout, the 20 mm BB-8 had 4% failure rate, the 25 mm BB-8 had 2% failure rate and the X tendobutton had 29% failure rate.
2. Between the 15 mm, 20 mm and 25 mm buttons the Bridge buttons with 8 mm bridge consistently performed far better than the buttons with 2 mm bridge (see table) proving that the bridge in the implant design is important for easy, predictable and secure locking when the buttons were passed serially 100 times and locked in the same 10 mm blowout hole in a bone model p<0.0005. Therefore the presence of bridge of 8 mm in the implant design is critical to the performance of the implant in 10 mm blowout holes.
3. In the 5 & 6 mm tunnel (n=20) there was almost 100% success rate for all buttons and all buttons performed well indicating that length of 15 mm and bridge of 2 mm was sufficient in accordance with the cover and side to side movement formula presented in the text: (in the 6 mm tunnel the 15 mm button provides a cover of 15−6/2=4.5 mm on each side; the side to side movement permitted is 6−2/2=2. The cover of 4.5 being greater than the side to side movement permitted of 2 mm allows the success of the smallest 15 mm button with 2 mm bridge. Similarly in a 7 or 8 mm blowout all the buttons may work but the failure of the 15 mm BB-2 is increased to 25% and 65% respectively signifying that the 15 mm BB-2 may not be advisable in blowouts more than 7 mm although it may lock at the inferior narrow end of the exit hole.
4. The Xtendobutton's® performance was similar to the 20 mm BB-2 button expectedly as the bridge of both the buttons was similar; both having 9% failure rate in the 9 mm blowout hole (n=100) and 29% failure in the knee model study and 65% failure (n=100) in the femur model study in a 10 mm blowout hole.
5. The 20 mm BB-8 had only 3% failure for 9 mm blowout hole and 4% failure for 10 mm blowout hole in the knee model study.
6. The performance of the 25 m BB-8 was the best approaching 99.5% success and surpassed all the buttons including the Xtendobutton in 5 to 10 mm tunnel blowouts.
7. No significant difference was noted with graft substitute or with single loop of ethibond no 2 passed through the loop. This proves that the graft has no contributory role or stabilizing/centralising effect on the button-loop complex in terms of correct locking and placement. It was also observed that even with graft inside the tunnel the buttons with low bridge displayed potential for side to side movement and could be rotated 360 degrees manually even after loading.
8. The 20 mm BB-8 showed success rate of 96% and failure rate of only 4% in 10 mm tunnel in the knee model study inspite of the fact that it had the same length of 20 mm as the XTB/20 MM BB-2 which showed a success rate of 71% and failure rate of 29%. This proves beyond any doubts that the buttons with longer bridge of 8 mm perform better in terms of reliable, reproducible and correct secure locking as well as placement and thus the Hypothesis of Bridge principle is validated.
9. The XTB showed 29% failure with graft and 34% failure with ethibond thread. This was comparable with the 20 mm BB-2 (36% failure with ethibond thread) precisely because of the similar bridge between the two.
10. The 20 mm BB-8 showed success rate of 96% and failure rate of only 4% inspite of the fact that it had the same length of 20 mm as the XTB/20 MM BB-2. This proves beyond any doubts that the buttons with longer bridge of 8 mm perform better in terms of reliable, reproducible and correct secure locking as well as placement and thus the Hypothesis Bridge principle is validated.
11. The 25 mm BB-2 showed a failure rate of 9% and success rate of 91% owing to its length; However due to the small bridge of 2 mm it displayed the potential for side to side movement.
12. The 25 mm BB-8 showed more than 98% success and resisted side to side movement.

The superior results of the knee model study as compared to the earlier bone model study clearly indicate that the direction of pull of the graft through the tibia after flipping contributes significantly to the successful locking and placement of the buttons since the graft is pulled in a downward and medial direction forcing the button in an anterior and inferior direction locking at the inferior narrow end of the tunnel. The presence of the graft inside the tunnel does not contribute significantly to the correct placement of the buttons.

In yet another study, the regular 12 mm Endobutton® was tried on bone model with 4.5 mm tunnel. Contrary to common belief, the flipping distance for this Endobutton® was hardly 5 to 6 mm and NOT 10 mm as described in many techniques (also mentioned in the product catalogue). At zero excess depth, more than half button is already out of the tunnel because the loop length corresponds to the tunnel length from the centre of the Endobutton® and NOT from its end or leading tip. Thus, at zero excess depth half of the button must project out by default. This knowledge is of critical importance in cases especially in anatomic ACL reconstruction where the tunnel length is small; e.g. if the tunnel length is 30 mm and 20 mm graft insert is desired, an excess depth of 10 mm can blow out the lateral cortex making Endobutton® fixation impossible. Whereas, only 5 mm excess depth is required for flipping the Endobutton®; thus an excess depth up to 25 mm in a 30 mm tunnel can make locking of the Endobutton® possible. Thus this knowledge is important as a preventive measure.

FIG. 21a illustrates a graphical view which compares EB, BB, and XTB for a 10 mm tunnel in terms of percentage of success.

FIG. 21b illustrates a graphical view which compares EB, BB, and XTB in terms of percentage of success.

FIG. 21c illustrates a graphical view which compares EB, BB, and XTB for a 10 mm tunnel in terms of percentage of success.

FIG. 21d illustrates a graphical view which compares EB, BB, and XTB in terms of percentage of success.

While considerable emphasis has been placed herein on the particular features of this invention, it will be appreciated that various modifications can be made, and that many changes can be made in the preferred embodiments without departing from the principles of the invention. These and other modifications in the nature of the invention or the preferred embodiments will be apparent to those skilled in the art from the disclosure herein, whereby it is to be distinctly understood that the foregoing descriptive matter is to be interpreted merely as illustrative of the invention and not as a limitation.

1. A bridge button formed by an elongate body of pre-defined dimensions such that said button being adapted to sit across a cavity in a bone, said button comprising a span member, located substantially at the centre of said elongate body, adapted to define a bridge portion, said span member being defined by lateral edges of sutures placed across adjacent inner circumferences of a pair of pre-determined holes used for passing suture loop, along a longitudinal axis of said bridge button, for holding a graft.

2. A bridge button as claimed in claim 1 wherein, said span member is a solid member, located substantially at the centre of said elongate body of said button, adapted to define a bridge portion.

3. A bridge button as claimed in claim 1 wherein, said span member is defined in accordance with the cavity it is adapted to sit across.

4. A bridge button as claimed in claim 1 wherein, said span member includes a plurality of holes for various purposes apart other than passing sutures for holding the graft.

5. A bridge button as claimed in claim 1 wherein, said span member includes a plurality of holes at pre-determined locations.

6. A bridge button as claimed in claim 1 wherein, said bridge button comprises a plurality of holes at pre-determined locations, beyond said bridge, adapted to allow passing of sutures for holding said graft, sutures for handling said button, sutures for handling an insert and the like purposes such that said holes may be selected in various combinations.

7. A bridge button as claimed in claim 1 wherein, said bridge button comprises a first laterally located hole at a first longitudinal edge of the button for passing sutures which act as the pulling suture, said pulling suture aiding in pulling said button along with graft through a formed tunnel.

8. A bridge button as claimed in claim 1 wherein, said bridge button comprises a second laterally located hole at a second longitudinal edge (longitudinal diametrically opposite to said first longitudinal edge) of the button used for passing sutures which act as the flipping suture, said flipping suture being engaged, by the surgeon, once the button has been pulled out of the tunnel on the lateral side of the bone, aiding in flipping (configuring) button from its operative vertical position (for pulling purposes) to its operative horizontal position wherein it can rest upon the cavity, transversely.

9. A bridge button as claimed in claim 1 wherein, said button comprises holes placed on either side of said span element thereby providing the bridge formed by the span element for providing a salvaging component in lateral femoral cortex blowout conditions in ACL or PCL reconstruction.

10. A bridge button as claimed in claim 1 wherein, said button comprises, two holes, equidistant from the centre, on either side, of said span element, said two holes being used to pass a suture loop which provides a support loop, on either side, for the ligament graft.

11. A bridge button as claimed in claim 1 wherein, said button comprises, two pairs of holes, equidistant respectively from the centre, on either side, of said span element, said two pairs of holes being used to pass a suture loop, on either side, which provides a pair of support loops for a pair of ligament grafts respectively.

12. A bridge button as claimed in claim 1 wherein, said holes include a transverse diametric rod member.

13. A bridge button formed by an elongate body of pre-defined dimensions such that said button being adapted to sit across a cavity in a bone, said button comprising a span member, located substantially at the centre of said elongate body, adapted to define a bridge portion, said span member comprising an under-collar which is a protrusion of pre-defined height and pre-defined length in accordance with the bridge principle, said protrusion being located substantially at the centre of the anchorage device or button, said under-collar comprising lateral walls which are orthogonal to the operative bottom surface of said button.

14. A bridge button as claimed in claim 13 wherein, at least a hole is provided on the protrusion of said button.

15. A bridge button as claimed in claim 13 wherein, at least a hole is provided on the shoulders of said button.

16. A bridge button as claimed in claim 13 wherein, said span member is a solid member, located substantially at the centre of said elongate body of said button, adapted to define a bridge portion.

17. A bridge button as claimed in claim 13 wherein, said span member is defined in accordance with the cavity it is adapted to sit across.

18. A bridge button as claimed in claim 13 wherein, said span member includes a plurality of holes for various purposes apart other than passing sutures for holding the graft.

19. A bridge button as claimed in claim 13 wherein, said span member includes a plurality of holes at pre-determined locations.

20. A bridge button as claimed in claim 13 wherein, said bridge button comprises a plurality of holes at pre-determined locations, beyond said bridge, adapted to allow passing of sutures for holding said graft, sutures for handling said button, sutures for handling an insert and the like purposes such that said holes may be selected in various combinations.

21. A bridge button as claimed in claim 13 wherein, said bridge button comprises a first laterally located hole at a first longitudinal edge of the button for passing sutures which act as the pulling suture, said pulling suture aiding in pulling said button along with graft through a formed tunnel.

22. A bridge button as claimed in claim 13 wherein, said bridge button comprises a second laterally located hole at a second longitudinal edge (longitudinal diametrically opposite to said first longitudinal edge) of the button used for passing sutures which act as the flipping suture, said flipping suture being engaged, by the surgeon, once the button has been pulled out of the tunnel on the lateral side of the bone, aiding in flipping (configuring) button from its operative vertical position (for pulling purposes) to its operative horizontal position wherein it can rest upon the cavity, transversely.

23. A bridge button as claimed in claim 13 wherein, said button comprises holes placed on either side of said span element thereby providing the bridge formed by the span element for providing a salvaging component in lateral femoral cortex blowout conditions in ACL or PCL reconstruction.

24. A bridge button as claimed in claim 13 wherein, said button comprises, two holes, equidistant from the centre, on either side, of said span element, said two holes being used to pass a suture loop which provides a support loop, on either side, for the ligament graft.

25. A bridge button as claimed in claim 13 wherein, said button comprises, two pairs of holes, equidistant respectively from the centre, on either side, of said span element, said two pairs of holes being used to pass a suture loop, on either side, which provides a pair of support loops for a pair of ligament grafts respectively.

26. A bridge button as claimed in claim 13 wherein, said holes include a transverse diametric rod member.

27. A bridge button formed by an elongate body of pre-defined dimensions such that said button being adapted to sit across a cavity in a bone, said button comprising a span member, located substantially at the centre of said elongate body, adapted to define a bridge portion, said span member comprising an under-collar which is a protrusion of pre-defined height and pre-defined length in accordance with the bridge principle, said protrusion being located substantially at the centre of the anchorage device or button, said under-collar comprising lateral walls which are orthogonal to the operative bottom surface of said button, said button still further comprising a recessed operative top surface, said recess being co-axial to any external button housing a graft.

28. A bridge button as claimed in claim 27 wherein, at least a hole is provided on the protrusion of said button.

29. A bridge button as claimed in claim 27 wherein, at least a hole is provided on the shoulders of said button.

30. A bridge button as claimed in claim 27 wherein, said span member is a solid member, located substantially at the centre of said elongate body of said button, adapted to define a bridge portion.

31. A bridge button as claimed in claim 27 wherein, said span member is defined in accordance with the cavity it is adapted to sit across.

32. A bridge button as claimed in claim 27 wherein, said span member includes a plurality of holes for various purposes apart other than passing sutures for holding the graft.

33. A bridge button as claimed in claim 27 wherein, said span member includes a plurality of holes at pre-determined locations.

34. A bridge button as claimed in claim 27 wherein, said bridge button comprises a plurality of holes at pre-determined locations, beyond said bridge, adapted to allow passing of sutures for holding said graft, sutures for handling said button, sutures for handling an insert and the like purposes such that said holes may be selected in various combinations.

35. A bridge button as claimed in claim 27 wherein, said bridge button comprises a first laterally located hole at a first longitudinal edge of the button for passing sutures which act as the pulling suture, said pulling suture aiding in pulling said button along with graft through a formed tunnel.

36. A bridge button as claimed in claim 27 wherein, said bridge button comprises a second laterally located hole at a second longitudinal edge (longitudinal diametrically opposite to said first longitudinal edge) of the button used for passing sutures which act as the flipping suture, said flipping suture being engaged, by the surgeon, once the button has been pulled out of the tunnel on the lateral side of the bone, aiding in flipping (configuring) button from its operative vertical position (for pulling purposes) to its operative horizontal position wherein it can rest upon the cavity, transversely.

37. A bridge button as claimed in claim 27 wherein, said button comprises holes placed on either side of said span element thereby providing the bridge formed by the span element for providing a salvaging component in lateral femoral cortex blowout conditions in ACL or PCL reconstruction.

38. A bridge button as claimed in claim 27 wherein, said button comprises, two holes, equidistant from the centre, on either side, of said span element, said two holes being used to pass a suture loop which provides a support loop, on either side, for the ligament graft.

39. A bridge button as claimed in claim 27 wherein, said button comprises, two pairs of holes, equidistant respectively from the centre, on either side, of said span element, said two pairs of holes being used to pass a suture loop, on either side, which provides a pair of support loops for a pair of ligament grafts respectively.

40. A bridge button as claimed in claim 27 wherein, said holes include a transverse diametric rod member.
